(19) United States
(12) Patent Application Publication
(10) Pub. No.: US 2021/0378859 A1
(43) Pub. Date: Dec. 9, 2021

(54) Heat Stimulation Instrument

(71) Applicant: ELLE CO., LTD., Tokyo (JP)

(72) Inventor: Hiroshi Nakamura, Tokyo (JP)

(21) Appl. No.: 17/337,867

(22) Filed: Jun. 3, 2021

(30) Foreign Application Priority Data
Jun. 4, 2020 (JP) 2020-097740

(51) Int. Cl. A61F 7/00

(52) U.S. Cl. CPC A61F 7/0085; A61F 2007/0087; A61F 2007/0062; A61F 7/007

(57) ABSTRACT

Provided is an instrument for applying heat stimulus to a target site in a human body in a convenient and efficient manner without causing trauma in a subject. A heat stimulation instrument includes a handle and a body unit, and the body unit includes an electric heater. The handle includes a vapor switch for controlling discharge of vapor, the body unit further includes a vaporizing chamber and a vapor chamber in fluid communication with the vaporizing chamber, the vapor chamber is provided with a steam expelling port passing through a bottom surface of the body unit, and the vapor switch controls such that steam passes through the vapor chamber and is discharged from the steam expelling port, the steam being formed from liquid introduced into the vaporizing chamber by being heated by the electric heater.

### CROSS-REFERENCES TO RELATED APPLICATIONS

This application claims the benefit of Japanese Patent Application No. 2020-097740, filed Jun. 4, 2020, which is hereby incorporated by reference in its entirety.

### BACKGROUND OF THE DISCLOSURE

### Field of the Disclosure

The present disclosure relates to an instrument for delivering heat stimulation on a human body, and in particular, to an instrument for delivering heat stimulation on the vagus nerve.

### Description of Related Art

Against a bodily slump of human, it has been a common practice to externally stimulate the corresponding site such as an affected area for activation and improvement by using various methods.

For example, International Publication No. WO 2010-106823 discloses that electric stimulus is applied to the vagus nerve for treatment of circulatory diseases such as acute myocardial infarction. In addition, for treatment intended to improve autonomic nervous activities of a patient into a suitable condition or improve an autonomic nervous disorder by conditioning activities of the sympathetic nervous system or the vagus nervous system, International Publication No. WO 2006-080075 discloses an autonomic nerve treatment device with a pressure loading means attached on the neck of a patient to load the neck with a suction force (negative pressure) or a pressing force (positive pressure) by the pressure loading means such that vagus nervous activities are increased or reduced and sympathetic nervous activities are reduced or increased to control sympathetic nervous activity values and/or vagus nervous activity values into target values for improving the autonomic nervous disorder.

However, the method described in International Publication No. WO 2010-106823 requires a surgical operation such as for embedding an electrode near the vagus nerve due to a need for directly applying electric stimulus on the vagus nerve. Further, the treatment device described in International Publication No. WO 2006-080075 applies pressure on the neck, which involves a large risk.

### SUMMARY OF THE DISCLOSURE

In view of the situation, an object of the present disclosure is to provide an instrument for applying heat stimulus to a target site in a human body in a convenient and efficient manner without causing trauma in a subject.

An aspect of the present disclosure is a heat stimulation instrument including a handle and a body unit, wherein the body unit includes an electric heater.

In another aspect of the present disclosure, the handle includes a vapor switch for controlling discharge of vapor,

the body unit further includes a vaporizing chamber and a vapor chamber in fluid communication with the vaporizing chamber,

the vapor chamber is provided with a steam expelling port extending through a bottom surface of the body unit, and

the vapor switch controls such that steam passes through the vapor chamber and is discharged from the steam expelling port, the steam being formed from liquid introduced into the vaporizing chamber by being heated by the electric heater.

According to the aspects of the present disclosure, it is possible to effectively activate the vagus nerve of the subject person to abate or heal various disease symptoms.

### BRIEF DESCRIPTION OF THE DRAWINGS

FIG. 1A is a sectional view of a heat stimulation instrument of the present disclosure;

FIG. 1B is a bottom view of the heat stimulation instrument; and

FIG. 2 illustrates a heat stimulation instrument according to another example of the present disclosure.

### DETAILED DESCRIPTION

An embodiment of a heat stimulation instrument according to the present disclosure will now be described in detail with reference to the drawings.

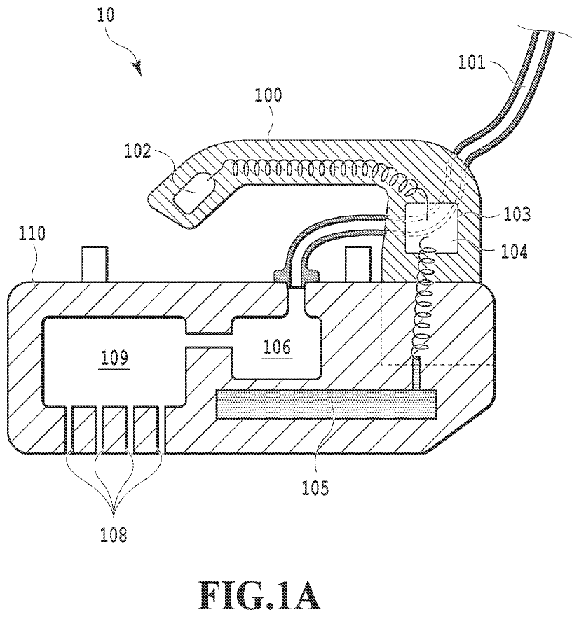

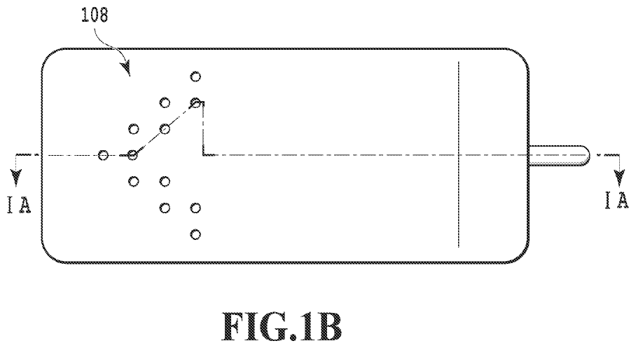

In an embodiment of the present disclosure, a heat stimulation instrument 10 includes a handle 100 provided with a vapor switch 102, and a body unit 110 that includes an electric heater 105, a vaporizing chamber 106, and a vapor chamber 109 in fluid communication with the vaporizing chamber 106 (See FIGS. 1A and 1B).

The handle 100 is provided with the vapor switch 102 and provided on top of the body unit 110. A user grips the handle 100, moves the body unit 110, and operates the vapor switch 102 to actuate an opening/closing-vapor chamber device 104 connected to a circuit board 103. The vapor switch 102 may be provided on any location on the handle 100.

The body unit 110 includes an electric heater 105, a vaporizing chamber 106, and a vapor chamber 109 in fluid communication with the vaporizing chamber 106, and is provided with a steam expelling port 108 extending from the vapor chamber 109 to the bottom surface of the body unit 110.

The vaporizing chamber 106 is provided adjacent to or near the electric heater 105 and is a space in which water introduced into the space is heated by the electric heater 105 into steam. A flow channel for introducing water is connected to the vaporizing chamber 106, and the vaporizing chamber 106 is in fluid communication with a dripping water flow channel 101 illustrated in FIG. 1A and a water reservoir (not illustrated). The water reservoir may be provided within the body unit 110.

The vapor chamber 109 is in fluid communication with the vaporizing chamber 106 and stores the steam formed in the vaporizing chamber 106.

Further, the steam expelling port 108 extending from the vapor chamber 109 to the bottom surface of the body unit 110 is provided and the vapor in the vapor chamber 109 is to be discharged from the bottom surface of the body unit 110. There may be provided a single steam expelling port 108 or a plurality of steam expelling ports 108. It is preferable to have a plurality of steam expelling ports 108 because a relatively large area can be heated simultaneously.

The steam is discharged when the vapor on/off device 104 is operated by the vapor switch 102. When an OPEN signal is transmitted from the vapor switch 102 to the circuit board 103, the opening/closing-vapor chamber device 104 is operated and the steam in the vapor chamber 109 is discharged from the steam expelling port 108.

In the embodiment of the present disclosure, the user provides heat stimulus to a subject site while pressing the body unit 110 against the site for pressurization. Although the pressure depends on the subject site, the heat stimulus may be provided while pressing is performed at a pressure on the order of, for example, 5 kPa to 15 kPa. A cushioning material such as a towel may be placed between the subject site and the body unit 110 when the body unit 110 is pressed against the subject site. Although depending on the subject site and its area for therapy delivery, the pressing may be performed several tens of times during the therapy delivery while pressing positions are being changed.

In pressing stimulation, the vapor is provided such that the temperature applied to the subject site is in a range of 35° C. to 60° C. through the cushioning material intervening therebetween. To provide the vapor, the temperature of the instrument bottom surface of the heat stimulation instrument 10 may be heated to 130° C. to 140° C.

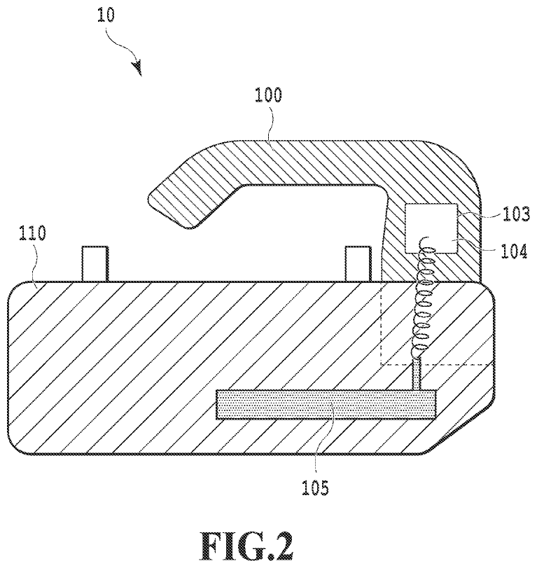

In another embodiment of the present disclosure, a heat stimulation instrument 10 includes a handle 100 and an electric heater 105, and is capable of providing heat stimulus by applying heat and pressure to a subject site. They will now be summarized in this order (See FIG. 2).

The handle 100 is provided on top of a body unit 110, and a user grips the handle 100 and moves the body unit 110.

The body unit 110 includes an electric heater 105. The body unit 110 is to be heated by the electric heater 105 and heated at such a temperature that the temperature in a range of 35 to 60° C. is applied to the subject site. In the embodiment of the present disclosure, a practitioner delivers therapy by placing a cushioning material such as a towel between the electric heater 105 and the subject site. Although depending on the subject site, as with the case of the embodiment with vapor, several tens of times of heat stimulation may be provided during the therapy delivery while pressing is performed at a pressure on the order of, for example, 5 kPa to 15 kPa. A cushioning material such as a towel may be placed between the subject site and the body unit 110 when the body unit 110 is pressed against the subject site.

In the embodiments of the present disclosure, the heat stimulation instrument 10 can provide heat stimulus to any site of a human body subjected to therapy. For example, heat stimulus can be provided to a back, an abdomen, a flank, a shoulder, an arm, a lumbar part, and a leg. The heat stimulation instrument 10 not only applies heat stimulus to a surface, but also provides stimulus to a deep part of the human body. For example, the vagus nerve in the back can be activated by providing heat stimulus to the back. Further, the heat stimulation instrument 10 can be used not only in medical applications, but also in cosmetic and bodywork applications.

Results of heat stimulation treatments performed on subject sites by using the heat stimulation instrument according to the embodiment of the present disclosure will now be illustrated.

### Example 1

Heat stimulation treatment was provided to a subject person (a woman in her 40s) who was suffering from flu symptoms and neck pain by using the heat stimulation instrument and performing several tens of times of pressing on her shoulder and back at a pressure on the order of 5 kPa to 15 kPa while generating vapor at a body unit temperature of 130 to 140° C. (subject site temperature 40° C.).

After the therapy delivery, her flu symptoms and neck pain were erased. In addition, after the therapy delivery compared with before the therapy delivery, reductions of −0.9 cm in her thigh, −0.5 cm in her calf, and −1.2 cm in her ankle were measured.

### Example 2

Heat stimulation treatment was provided to a subject person (a man in his 60s) who was suffering from symptoms of lumbar pain, back pain, and muscle stiffness of shoulder by using the heat stimulation instrument and performing 30 times of pressing on each of his shoulder, back, lumbar part, and hip at a pressure on the order of 5 kPa to 15 kPa while generating vapor at a body unit temperature of 130 to 140° C. (subject site temperature 40° C.).

After the therapy delivery, his lumbar pain and back pain were erased. In addition, after the therapy delivery compared with before the therapy delivery, differences were found in anteflexion and postexion, and flexibility of his body was improved. The results are shown in Table 1.

TABLE 1

| | Anteflexion | Postexion |
|---|---|---|
| Before therapy delivery | +5.5 cm | 129 cm above floor |
| After first therapy delivery | +1.5 cm | 122 cm above floor |
| After second therapy delivery | −4.3 cm | 117 cm above floor |
| Variation | −9.8 cm | −12 cm |

### Example 3

Heat stimulation treatment was provided twice to a subject person (a man in his 60s) who was injured in a fall by using the heat stimulation instrument and performing several tens of times of pressing on his shoulder and back at a pressure on the order of 5 kPa to 15 kPa while generating vapor at a body unit temperature of 130 to 140° C. (subject site temperature 40° C.).

After the therapy delivery compared with before the therapy delivery, differences were found in anteflexion and postexion, and flexibility of his body was improved. The results are shown in Table 2.

TABLE 2

| | Anteflexion | Postexion |
|---|---|---|
| Before therapy delivery | +5.0 cm | 120 cm above floor |
| After first therapy delivery | +0.3 cm | 115 cm above floor |
| After second therapy delivery | −1.2 cm | 113 cm above floor |
| Variation | −6.2 cm | −7 cm |

### Example 4

Heat stimulation treatment was provided twice to a subject person (a man in his 60s) who was suffering from left neck pain by using the heat stimulation instrument and performing several tens of times of pressing on his back left and right at a pressure on the order of 5 kPa to 15 kPa while generating vapor at a body unit temperature of 130 to 140° C. (subject site temperature 40° C.).

After therapy delivery, the excursion of his neck was improved. In addition, after the therapy delivery compared with before the therapy delivery, differences were found in anteflexion and postexion, and flexibility of his body was improved. The results are shown in Table 3.

TABLE 3

| | Anteflexion | Postexion |
|---|---|---|
| Before therapy delivery | +9.0 cm | 130 cm above floor |
| After first therapy delivery | +1.5 cm | 128 cm above floor |
| After second therapy delivery | −3.0 cm | 25 cm above floor |
| Variation | 12.0 cm | −5 cm |

As described above, in the embodiment of the present disclosure, the heat stimulation instrument could activate the vagus nerve, remove bodily slump, and improve bodily flexibility.

The present disclosureis to be construed only from matters described in claims, and in addition to the matters, any alterations and modifications that may be encompassed within the concepts of the present disclosuremay be made to the above-described embodiments. In other words, all matters in the above-described embodiments are not intended to limit the present disclosureand may be subjected to any change depending on applications and purposes thereof, including any and all configurations that are not in direct association with the present disclosure.

What is claimed is:

1. A heat stimulation instrument comprising:
a handle and a body unit, wherein
the body unit includes an electric heater.

2. The heat stimulation instrument according to claim 1, wherein
the handle includes a vapor switch for controlling discharge of vapor,
the body unit further includes a vaporizing chamber and a vapor chamber in fluid communication with the vaporizing chamber,
the vapor chamber is provided with a steam expelling port extending through a bottom surface of the body unit, and
the vapor switch controls such that steam passes through the vapor chamber and is discharged from the steam expelling port, the steam being formed from liquid introduced into the vaporizing chamber by being heated by the electric heater.

3. The heat stimulation instrument according to claim 2, wherein the introduced liquid is water.

4. The heat stimulation instrument according to claim 1, wherein the body unit is at 50° C. or higher.

5. The heat stimulation instrument according to claim 2, wherein the body unit is at 50° C. or higher.

6. The heat stimulation instrument according to claim 3, wherein the body unit is at 50° C. or higher.

7. The heat stimulation instrument according to claim 4, wherein the body unit is at 140° C. or lower.

8. The heat stimulation instrument according to claim 5, wherein the body unit is at 140° C. or lower.

9. The heat stimulation instrument according to claim 6, wherein the body unit is at 140° C. or lower.

10. The heat stimulation instrument according to claim 4, wherein the body unit is heated such that a subject site is subjected to therapy at a temperature of 35 to 60° C.

11. The heat stimulation instrument according to claim 5, wherein the body unit is heated such that a subject site is subjected to therapy at a temperature of 35 to 60° C.

12. The heat stimulation instrument according to claim 6, wherein the body unit is heated such that a subject site is subjected to therapy at a temperature of 35 to 60° C.

13. The heat stimulation instrument according to claim 7, wherein the subject site is pressed for being subjected to therapy at a pressure of 5 kPa to 15 kPa.

14. The heat stimulation instrument according to claim 10, wherein the subject site is pressed for being subjected to therapy at a pressure of 5 kPa to 15 kPa.

15. A method of applying heat stimulus to a subject, comprising:
providing a heat stimulation instrument including a handle and a body unit, the body unit including an electric heater;
heating the body unit to 50° C. or higher; and
pressing the heated heat stimulation instrument against the subject.

16. The method according to claim 15, wherein the body unit is heated to 140° C. or lower.

17. The method according to claim 15, wherein the subject site is pressed for being subjected to therapy at a pressure of 5 kPa to 15 kPa.

18. The method according to claim 15, wherein the body unit is heated such that a subject site is subjected to therapy at a temperature of 35 to 60° C.

19. The method according to claim 15, wherein
the handle includes a vapor switch for controlling discharge of vapor,
the body unit further includes a vaporizing chamber and a vapor chamber in fluid communication with the vaporizing chamber,
the vapor chamber is provided with a steam expelling port extending through a bottom surface of the body unit, and
the pressing is performed while using the vapor switch to control such that steam passes through the vapor chamber and is discharged from the steam expelling port, the steam being formed from liquid introduced into the vaporizing chamber by being heated by the electric heater.
